(19) United States
(12) Patent Application Publication
(10) Pub. No.: US 2023/0295225 A1
(43) Pub. Date: Sep. 21, 2023

(54) 3D NEURONAL TISSUE GRAFTS USING ULTRASHORT SELF-ASSEMBLING PEPTIDE SCAFOLDS

(71) Applicant: KING ABDULLAH UNIVERSITY OF SCIENCE AND TECHNOLOGY, Thuwal (SA)

(72) Inventors: Charlotte A. E. HAUSER, Thuwal (SA); Sherin Abubaker ABDELRAHMAN, Thuwal (SA)

(21) Appl. No.: 18/021,596

(22) PCT Filed: Aug. 19, 2021
(86) PCT No.: PCT/IB2021/057625

§ 371 Date: Feb. 16, 2023

Related U.S. Application Data
(63) Continuation of application No. 17/401,434, filed on Aug. 13, 2021.
(60) Provisional application No. 63/067,913, filed on Aug. 20, 2020.
(60) Provisional application No. 63/067,923, filed on Aug. 20, 2020.

(51) Int. Cl. C07K 5/103; A61L 26/00; C08J 3/075; C08J 3/09; A61L 27/22; A61L 31/04; A61L 31/14; C12N 5/00; A61L 27/52

(52) U.S. Cl. CPC C07K 5/101; A61L 26/0028; A61L 26/008; C08J 3/075; C08J 3/09; A61L 27/22; A61L 31/043; A61L 31/145; C12N 5/0068; A61L 27/52; C12N 2513/00; A61K 38/00

(57) ABSTRACT

The present invention relates to a functional 3D neuronal model based on ultrashort self-assembling peptide scaffolds in accordance with the present invention, and to a method of preparing such a model. The models are suitable for in vitro drug testing, cellular replacement therapies as well as other applications.

### CROSS-REFERENCE TO RELATED APPLICATIONS

This application claims benefit of priority to U.S. Provisional Patent Application No. 63/067,923, entitled “3D NEURONAL TISSUE GRAFTS USING ULTRASHORT SELF-ASSEMBLING PEPTIDE SCAFFOLDS” filed Aug. 20, 2020; U.S. Provisional Patent Application No. 63/067,913 entitled, “PEPTIDE COMPOUND WITH REPETITIVE SEQUENCES” filed Aug. 20, 2020; and U.S. patent application Ser. No. 17/401,434 entitled “PEPTIDE COMPOUND WITH REPETITIVE SEQUENCES” filed Aug. 13, 2021. The entire contents and disclosures of these patent applications are incorporated herein by reference in their entirety.

### BACKGROUND

### Field of the Invention

The present disclosure relates to generally to a functional 3D neuronal model based on ultrashort self-assembling peptide scaffolds. The present disclosure further relates to a method of preparing and using such a 3D model.

### Background of the Invention

The brain is one of the most complex organs that is responsible of conducting critical biological functions including cognition, memory, speech and movement. Damage to any region of the brain may lead to deleterious health effects that may result in the development of neurodegenerative disorders such as dementia, Alzheimer or Parkinson's diseases. Studying such diseases requires the careful designing of an in vitro model that closely recapitulate the in vivo situation.

Many of the currently available neuronal models are 2D based systems were the cells are exposed directly to plastic surfaces on which they are cultivated which leads to their exposure to mechanical cues that are far different from the natural in vivo 3D system. On the other hand, majority of the available neuronal 3D models are based on scaffolds that are animal derived, infused with external chemicals like growth factors, requires the use of bioreactors for efficient delivery of nutrients, requires chemical or UV crosslinking, or requires tedious preparation steps that possess a challenge on reproducibility^{2,3}.

All those limitations make the available 2D and 3D systems not suitable for many applications such as cellular replacement therapies. Therefore, there exists a need for a new 3D model^{2,3.}

### SUMMARY

According to first broad aspect, the present disclosure provides a functional 3D neuronal model comprising of neurons embedded in ultrashort self-assembling peptide scaffolds.

According to a second broad aspect, the present disclosure provides a method of preparing such 3D models through 3D bioprinting that uses the mixture of neurons and ultrashort self-assembling peptide as bioinks.

An aspect the invention provides a tool for in vitro drug testing, neurology research platform and cellular replacement therapies for neurological disorders.

Other aspects and features of the present disclosure will become apparent to those skilled in the art upon review of the following description of specific embodiments of the invention in conjunction with the accompanying figures.

### BRIEF DESCRIPTION OF THE DRAWINGS

The patent or application file contains at least one drawing executed in color. Copies of this patent or patent application publication with color drawing(s) will be provided by the office upon request and payment of the necessary fee.

The accompanying drawings, which are incorporated herein and constitute part of this specification, illustrate exemplary embodiments of the invention, and, together with the general description given above and the detailed description given below, serve to explain the features of the invention.

FIG. 1 is a graph showing the cell viability of primary mouse cortical neurons within IIFK and IIZK peptide hydrogels according to an exemplary embodiment of the present invention.

FIG. 2 is a graph showing the expression of neuron-specific mouse anti-β-III tubulin (TUJ1) by primary mouse cortical neurons within IIFK and IIZK peptide hydrogels according to an exemplary embodiment of the present invention.

FIG. 3 is a graph showing the primary mouse cortical neurons proliferation through quantitation of ATP production according to an exemplary embodiment of the present invention.

FIG. 4 is a graph showing the analysis of TUJ1 positive neurons morphology according to an exemplary embodiment of the present invention.

FIG. 5 is a graph showing the immune-stained primary cortical neurons 3D cultured in 1 mg/ml IIZK peptide according to an exemplary embodiment of the present invention.

FIG. 6 is a graph showing the immune-stained primary cortical neurons 3D cultured in 2 mg/ml IIFK peptide according to an exemplary embodiment of the present invention.

FIG. 7 is a graph showing the differentiation of hESCs into cortical neurons in 1 mg/ml KIVAV peptide according to an exemplary embodiment of the present invention.

FIG. 8 is a graph showing the immune-stained primary cortical neurons 3D cultured in 5 mg/ml IVFR and 2 mg/ml IVZR peptides according to an exemplary embodiment of the present invention.

FIG. 9 is a graph showing the immune-stained primary cortical neurons 3D cultured in 5 mg/ml IVFR, 2 mg/ml IVZR and 1 mg/ml IIZK peptides according to an exemplary embodiment of the present invention.

FIG. 10 is a graph showing the cell viability of primary mouse cortical neurons 3D cultured in 2 mg/ml FFIK, 2 mg/ml IIFK and 1 mg/ml IIZK peptides according to an exemplary embodiment of the present invention.

FIG. 11 is a graph showing the extracellular electrical activity of mouse embryonic cortical neurons 3D cultured in 2 mg/ml IIFK and 1 mg/ml IIZK peptides according to an exemplary embodiment of the present invention.

### DETAILED DESCRIPTION OF THE INVENTION

### Definitions

Where the definition of terms departs from the commonly used meaning of the term, applicant intends to utilize the definitions provided below, unless specifically indicated.

Unless defined otherwise, all technical and scientific terms used herein have the same meaning as is commonly understood to which the claimed subject matter belongs. In the event that there is a plurality of definitions for terms herein, those in this section prevail. All patents, patent applications, publications and published nucleotide and amino acid sequences (e.g., sequences available in GenBank or other databases) referred to herein are incorporated by reference. Where reference is made to a URL or other such identifier or address, it is understood that such identifiers can change and particular information on the internet can come and go, but equivalent information can be found by searching the internet. Reference thereto evidences the availability and public dissemination of such information.

It is to be understood that the foregoing general description and the following detailed description are exemplary and explanatory only and are not restrictive of any subject matter claimed. In this application, the use of the singular includes the plural unless specifically stated otherwise. It must be noted that, as used in the specification and the appended claims, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

For purposes of the present disclosure, the term “comprising”, the term “having”, the term “including,” and variations of these words are intended to be open-ended and mean that there may be additional elements other than the listed elements.

For purposes of the present disclosure, directional terms such as “top,” “bottom,” “upper,” “lower,” “above,” “below,” “left,” “right,” “horizontal,” “vertical,” “up,” “down,” etc., are used merely for convenience in describing the various embodiments of the present disclosure. The embodiments of the present disclosure may be oriented in various ways. For example, the diagrams, apparatuses, etc., shown in the drawing figures may be flipped over, rotated by 90° in any direction, reversed, etc.

For purposes of the present disclosure, a value or property is “based” on a particular value, property, the satisfaction of a condition, or other factor, if that value is derived by performing a mathematical calculation or logical decision using that value, property or other factor.

For purposes of the present disclosure, it should be noted that to provide a more concise description, some of the quantitative expressions given herein are not qualified with the term “about.” It is understood that whether the term “about” is used explicitly or not, every quantity given herein is meant to refer to the actual given value, and it is also meant to refer to the approximation to such given value that would reasonably be inferred based on the ordinary skill in the art, including approximations due to the experimental and/or measurement conditions for such given value.

For purposes of the present invention, the term “gel” and “hydrogel” are used interchangeably. These terms refer to a is a network of polymer chains, entrapping water or other aqueous solutions, such as physiological buffers, of over 99% by weight. In an embodiment of the present invention, the polymer chains may be a peptide with repetitive sequences.

For purposes of the present disclosure, the term “ultra-short peptide” and “self-assembling peptide” are used interchangeably. These terms refer to a sequence containing 3-7 amino acids. The peptides according an aspect of the present disclosure are also particularly useful for formulating aqueous or other solvent compositions, which may be used for printing structures, in particular 3D structures. Such printed structures make use of the gelation properties of the peptides according to features of the present disclosure.

For purposes of the present disclosure, the term “bioinks” as used herein means materials used to produce engineered/artificial live tissue using 3D printing. In the present disclosure, these bioinks are mostly composed of hydrogel or organogel with cellular components embedded.

For purposes of the present disclosure, the term “scaffolds” as used herein means the ultra-short peptide or other polymer materials in the bioinks that provide support for the cellular components.

### Description

While the invention is susceptible to various modifications and alternative forms, specific embodiment thereof has been shown by way of example in the drawings and will be described in detail below. It should be understood, however that it is not intended to limit the invention to the particular forms disclosed, but on the contrary, the invention is to cover all modifications, equivalents, and alternatives falling within the spirit and the scope of the invention.

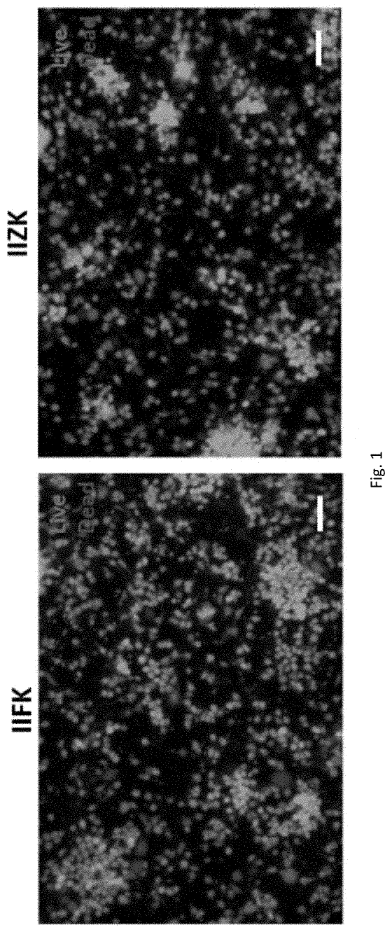

In one embodiment, mouse primary cortical neurons are 3D cultured within the 3D scaffold formed by peptide hydrogel comprising IIFK or IIZK peptides, and cell growth and properties are assessed against the gold standard Poly-D-Lysin (PDL). FIG. 1 shows the result of live/dead cells' viability assay of mouse primary cortical neurons stained with calcein-AM and ethidium homodimer-1. In FIG. 1, the green stained dots indicate live cells, while the red stained dots indicate dead cell. The left penal shows the viability of mouse primary cortical neurons cultured in IIFK scaffold, while the right penal shows the viability of mouse primary cortical neurons cultured in IIZK scaffold. Most cells are stained in green, with only a few red stained cells in FIG. 1. Thus, the mouse primary cortical neurons cultured in the hydrogel scaffold have high viability of at least 90%.

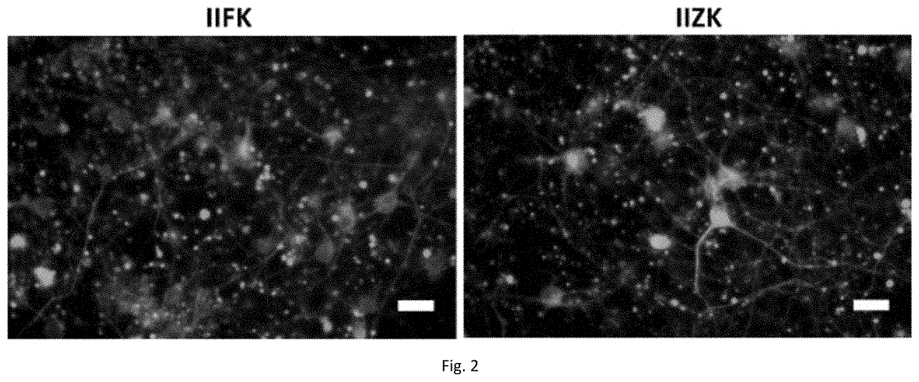

FIG. 2 shows the result of mouse cortical neurons labeled with neuron-specific mouse anti-β-III tubulin (TUJ1) antibodies and counterstained with DAPI. In FIG. 1, the green staining indicates the distribution of tubulin labelled with TUJ1, while the blue staining indicates the location of nucleus stained with DAPI. The left penal shows mouse primary cortical neurons cultured in IIFK scaffold, while the right penal shows mouse primary cortical neurons cultured in IIZK scaffold. The presence of neurite is visible in neurons cultured in both IIFK and IIZK scaffolds.

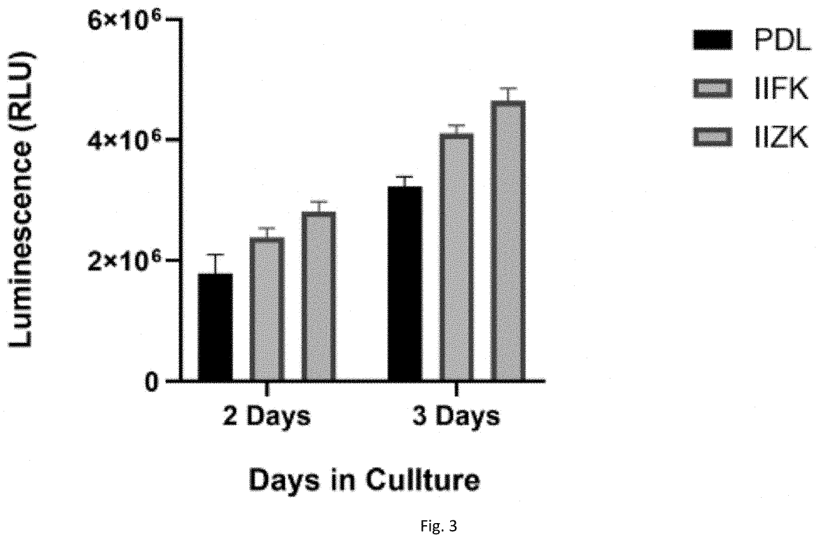

FIG. 3 shows the assessment of cell proliferation in comparison to poly-D-lysine (PDL) 2D cultures. The cell proliferation is directly proportional to the luminescence value. Thus, as shown in FIG. 3, neurons cultured in IIFK and IIZK scaffolds have higher proliferation level than those in poly-D-lysine (PDL) 2D cultures. Moreover, neurons cultured in IIZK scaffold have higher proliferation level than those in poly-D-lysine (PDL) 2D cultures IIFK scaffold.

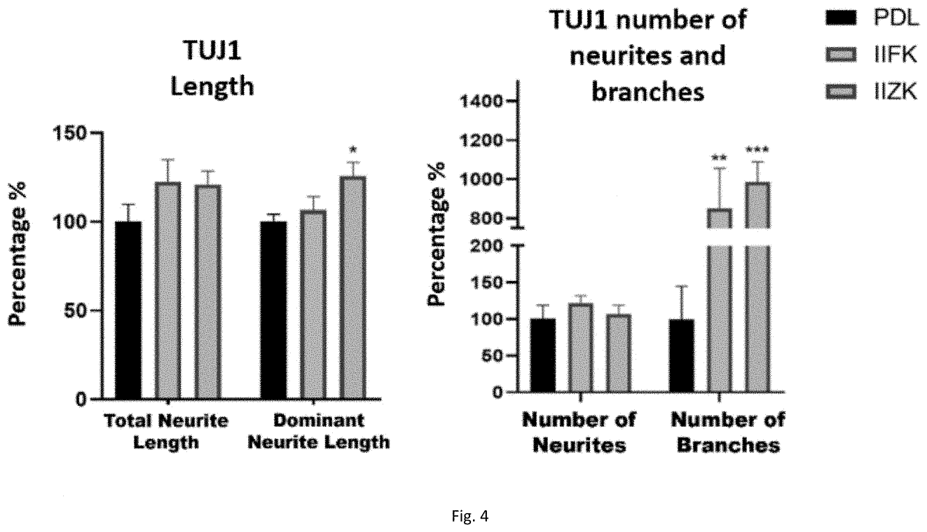

FIG. 4 shows analyses of TUJ1 positive neurons with respect to neurite numbers, length, and outgrowth (***p<0.001; **p<0.01; *p<0.05). Scale bar corresponds to 50 μm. As shown in FIG. 4, there is significant increase in the dominant neurite length and the number of neuron branches for cells 3D cultured in IIZK and IIFK in comparison to PDL, respectively. Interestingly, the observed increase in neurite length and number of branches is higher in neurons cultured in IIZK than in IIFK. This could be attributed to the fact that IIZK has lower rigidity (G′6.52±0.18 kPa) at the MGC when compared to IIFK (G′14.45±1.37 kPa).

The results show that the peptide hydrogel supports viability, and promotes maturation of primary neurons and neurite outgrowth.

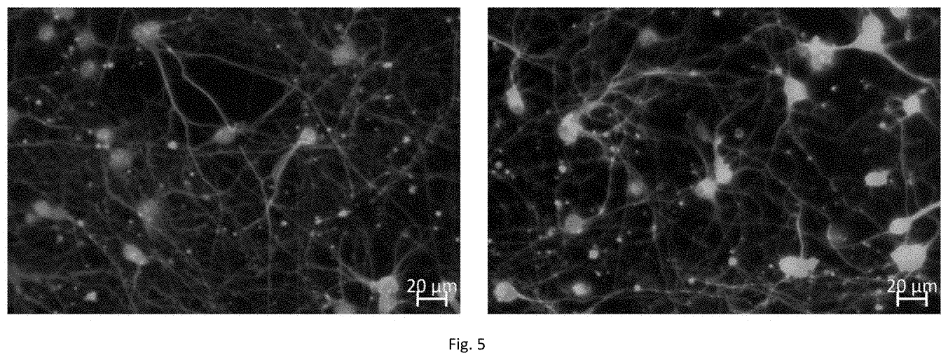

In one embodiment, primary cortical neurons are 3D cultured within 1 mg/ml IIZK peptide scaffold for 8 days. FIG. 5 shows the morphology of neurons after 8-day culture. The neurons are visualized by the neuronal marker stained in green fluorescence and the nucleus stained in blue fluorescence. The results show the primary cortical neurons presented normal morphology after being cultured in 3D in IIZK peptide scaffold.

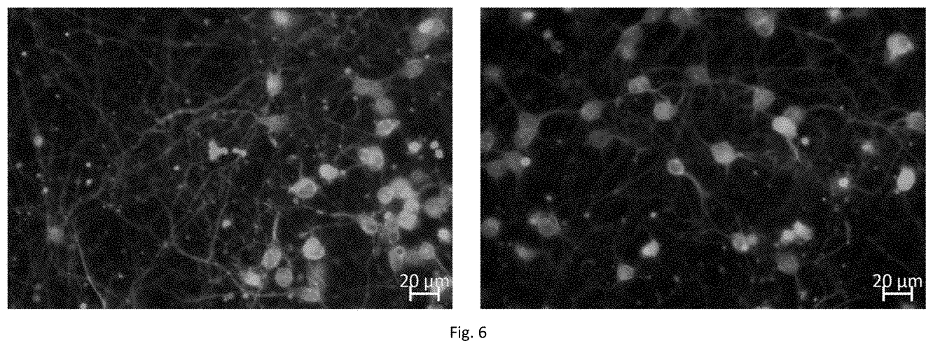

In one embodiment, primary cortical neurons are 3D cultured within 2 mg/ml IIFK peptide scaffold for 8 days. FIG. 6 shows the morphology of neurons after 8-day culture. The neurons are visualized by the neuronal marker stained in green fluorescence and the nucleus stained in blue fluorescence. The results show the primary cortical neurons presented normal morphology after being cultured in 3D in IIFK peptide scaffold.

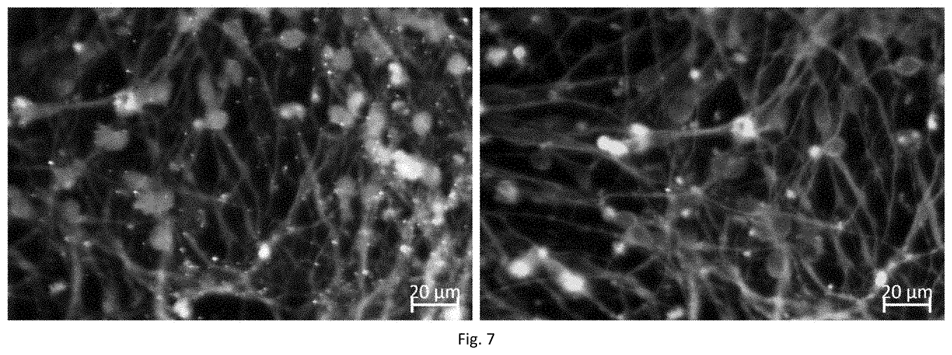

In one embodiment, human embryonic stem cells (hESCs) are 3D cultured within 1 mg/ml KIVAV peptide hydrogel for 33 days. FIG. 7 shows the morphology of cortical neurons differentiated from hESCs after 33-day culture. The morphology of neurons confirmed that the KIVAV peptide hydrogel supports the differentiation of cortical neurons.

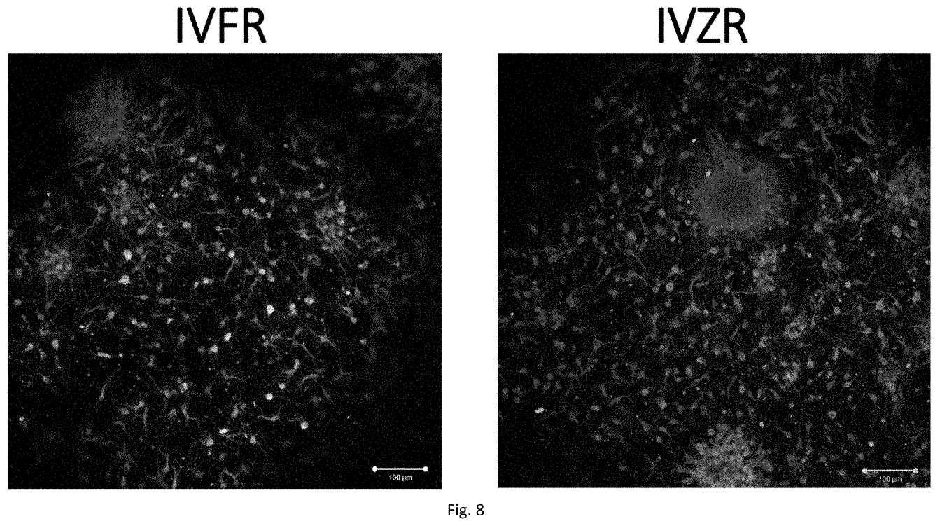

In one embodiment, primary mouse cortical neurons are 3D cultured within 5 mg/ml IVFR peptide hydrogel or 2 mg/ml IVZR peptide hydrogel. FIG. 8 shows the morphology of neurons after 3-day culture. The neurons are visualized by the neuronal marker stained in green fluorescence, T-Box Brain Transcription Factor 1 (TBR1) in red fluorescence and the nucleus stained in blue fluorescence. TBR1 is a protein expressed primarily in the cerebral cortex, hippocampus, amygdala and olfactory bulb. The results show the primary cortical neurons presented normal morphology and survived well after cultured in 3D in IVFR or IVZR peptide scaffold.

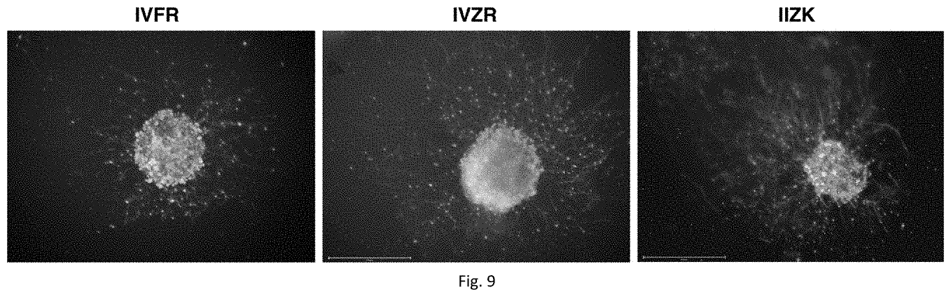

In one embodiment, primary mouse cortical neurons are 3D cultured within 5 mg/ml IVFR peptide hydrogel, 2 mg/ml IVZR peptide hydrogel or 1 mg/ml IIZK peptide hydrogel. FIG. 9 shows the migration of neurons out of the neurospheres toward the peptide surface after 5-day culture. The neurons are visualized by the neuronal marker stained in green fluorescence, T-Box Brain Transcription Factor 1 (TBR1) in red fluorescence and the nucleus stained in blue fluorescence. TBR1 is a protein expressed primarily in the cerebral cortex, hippocampus, amygdala and olfactory bulb. The results show the primary cortical neurons presented normal morphology and survived well after cultured in 3D in IVFR or IVZR peptide scaffold.

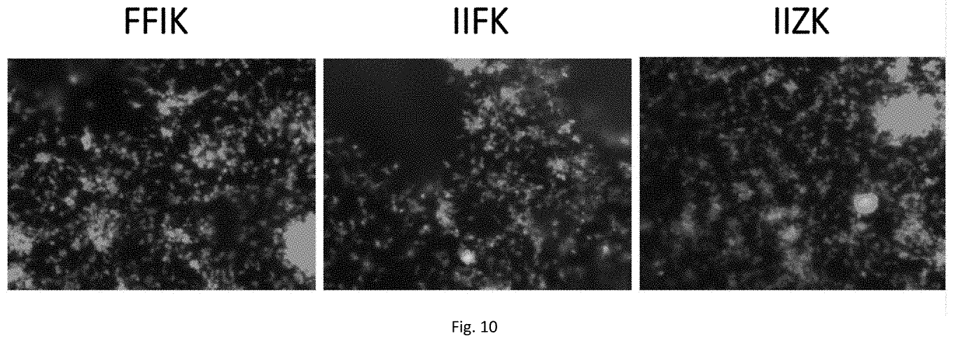

In one embodiment, primary mouse cortical neurons are 3D cultured within 2 mg/ml FFIK peptide hydrogel, 2 mg/ml IIFK peptide hydrogel or 1 mg/ml IIZK peptide hydrogel. As shown in FIG. 10, the results show that all three peptide hydrogels support viability of neurons.

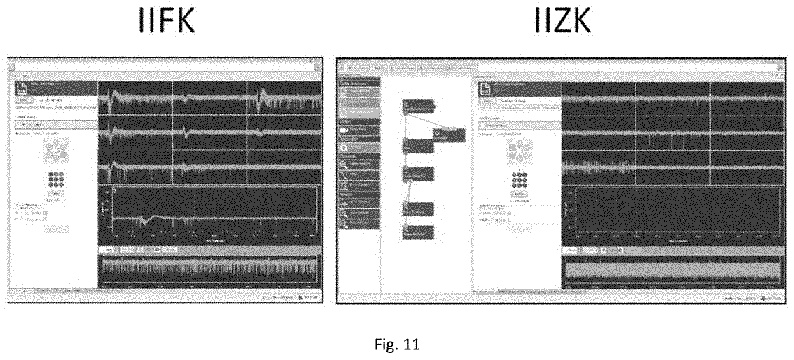

In one embodiment, primary mouse cortical neurons are 3D cultured within 2 mg/ml IIFK peptide hydrogel using multielectrode arrays (MEA). FIG. 11 shows the extracellular electrical activities.

In one embodiment, primary mouse cortical neurons are 3D cultured within 2 mg/ml IIZK peptide hydrogel using multielectrode arrays (MEA). FIG. 11 shows the extracellular electrical activities.

### EXAMPLES

### Example 1

### 3-Dimensional Neuronal Tissue Model

Primary mice neurons were isolated from the embryos of time mated Swiss mice at different embryonic stages. In order to establish the 3-dimensional neuronal tissue model, cell culture plates were first coated with a layer of peptide hydrogels in cell culture grade water. Then DPBS was added to a final concentration of 1× and plates were incubated at 37° C. for 10-15 minutes to promote hydrogel formation. This peptide base was first prepared in order to minimize possibility of cells contact or adherence to the plastic cell culture plate surface and to ensure the growth and proliferation of cells in 3D within the peptide scaffold. Cells in 1×PBS were then encapsulated within another layer of peptide in water that was added on top of the previously prepared peptide base and mixed carefully by swirling. This 3D construct was incubated for 10-15 minutes before adding freshly prepared N2 media. The media was composed of 1:1 mixture of F12 medium and Minimum Essential Medium (MEM), 1 mM glutamine, 1 mg/ml bovine serum albumin, 15 mM HEPES, 6 mg/ml glucose, 1% penicillin/streptomycin (P/S) and 1% N2 supplement Cells were incubated at 37° C. and 5% CO₂ before fixation and analysis.

### Example 2

### Immunofluorescence Staining of Primary Mice Cortical Neurons

Neurons were fixed by 4% paraformaldehyde (PFA). Media was removed carefully to avoid breaking the neurites and the cells were subsequently washed with 1× DPBS. An appropriate volume of 4% PFA was then added and left at room temperature for 5 minutes. Then it was removed and discarded followed by adding 1× DPBS. The cells were then kept at 4° C. until stained. Fixed cells were incubated with primary antibodies cocktail diluted in blocking buffer (5% FBS, 0.3% triton-X, and 0.2% sodium azide) overnight at room temperature. After removing and discarding the primary antibodies, cells were incubated for 1 hour at room temperature in blocking buffer. This was followed by adding secondary antibodies diluted in blocking buffer and incubating for 2 hours at room temperature. Subsequently, cells were incubated for 5 minutes with 1:2000 DAPI diluted in water then it was removed and discarded. Finally, 1xDPBS was added to each well and the plates were kept at 4° C. until analyzed. Imaging was then done using a ZEISS™ fluorescence microscope, Zen blue v 2.6 software, and/or laser scanning confocal microscope (Zeiss™ LSM 880 Inverted Confocal Microscope, Germany).

### Example 3

### Neurites Morphology Analysis

Neurites' morphology analysis was carried out on TUJ1 positive neurons. Ten neurons from each technical replicate were analyzed for total neurites length, dominant neurite length, number of neurites and, branches according to the method described by Blakely et al.⁴ Neurite tracing was done using NeuronJ plugin in ImageJ software v 1.51k to determine neurite length. Overlapping neurites and the ones shorter than 20 μm were excluded to avoid bias as in such cases, it was difficult to ascertain to which soma the corresponding neurite belonged. Data obtained from TUJ1 positive neurons 3D cultured in selected peptides were normalized to the control group for neurons cultured at the same time in Poly-D-Lysine (PDL). Subsequently, data were expressed as a percentage change from the control which was considered to be 100%. Images used for this analysis were obtained using ZEISS™ fluorescence microscope, Zen blue v 2.6 software on 20×objective. Student t-test was done using GraphPad prism v 8.1.2, and all quantitative data were expressed as means f SEMs with the significance being set at p<0.05.

### Example 4

### Neurospheres Migration Assay

Neurospheres were prepared by seeding at least 4 million primary neurons in 10 ml of N2 media on non-treated/non-adherent cell culture dish. Growth factors were added in 1:10000 concentration for EFG (1 ul/10 m) and 1:5000 FGF2 (2ul/10 ml). Plates were then kept in the incubator at 37° C. and 5% CO₂ for 5 days during which neurospheres formation was inspected on daily basis using inverted microscope. Freshly prepared EFG and FGF2 were added once every two days. At day 5, neurospheres were transferred to 15- or 50-ml falcon tube. The tube was left standing at room temperature for 30 minutes to allow the neurospheres to settle at the bottom of the tube forming a visible pellet. Cell culture media was then removed carefully leaving about 1 ml of media. Neurospheres were then resuspended in the remaining media and seeded within peptide hydrogel. Migration of neurons out of the spheres was observed daily for up to 5 days. After 5 days cells were fixed and stained with TUJ1, TUJ1 and TBR1, or TUJ1 and TH depending on the neuronal cells type.

### Example 5

Differentiation of Embryonic Stem Cells (ESCs) into Cortical Neurons

ESCs were seeded on peptide hydrogel in 48 well plates at a density of 5000 cells per well and incubated overnight in leukemia inhibitory factor (LIF) basic medium prior to starting the differentiation. 80 μL of peptide hydrogel construct was in each well of the 48-well plate. A combination of serum replacement medium (SRM) and N2 medium, supplemented with 200 nM LDN193189 (Tocris®), were used for early patterning stage under gradient conditions from day 0-7, (day 0: 100% SRM; day 1: 75% SRM:25% N2; day 2: 50% SRM:50% N2; day 3: 25%SRM:75% N2). Basic LIF medium consists of Knockout DMEM (GIBCO®), 15% fetal bovine serum (FBS, Sigma-Aldrich®), 1× P/S, 1× GlutaMAX™, 1× non-essential amino acids (NEAA), 0.11 mM beta-mercaptoethanol and 2000 IU/ml LIF (Millipore®). SRM consisted of Knockout DMEM, 1× P/S, 1× GlutaMAX™, 1× NEAA, 0.11 mM beta-mercaptoethanol and 15% knockout serum. N2 medium components included DMEM/F12, 1 × P/S, 1× GlutaMAX™, 1× NEAA, 0.11 mM beta-mercaptoethanol, 1× insulin-transferrin-selenium-sodium pyruvate supplement (ITS-A) and 1× N2 supplement. N2B27 medium was utilized for the maturation stage (day 7 to day 15). Maturation medium consisted of 1:1 mixture of DMEM/F12 and Neurobasal medium, 1× P/S, 1× GlutaMAX™, 1× NEAA, 0.11 mM beta-mercaptoethanol, 1× ITS-A, 1× N2 supplement and 1× B27+vitamin A supplement. For ventral midbrain specification, 200 ng/ml Sonic Hedgehog (Shh) (R&D), 2 uM purmorphamine (PM) (Stemgent®) and 25 ng/ml fibroblast growth factor 8 (FGF8) (R&D™) were added to the patterning media on day 1-190 7, and further supplemented with 0.3 uM CHIR9902 (Stemgent®) on day 2-7, as well as 20 ng/ml FGF2 (Peprotech®) on day 3-7. On day 7, the differentiated cells were split (1:1 ratio) using accutase to 48 wells plate coated with FC. The wells were coated as described earlier. From day 7-15 cell were switched to maturation medium (N2B27) plus 30 ng/ml glial cell-derived neurotrophic factor (GDNF) (R&D™), 30 ng/ml brain-derived neurotrophic factor (BDNF) (R&D™), 0.2 mM AA (Sigma-Aldrich®) and 10 uM N-[N-(3,5-Difluorophenacetyl)-L-alanyl]-S-phenylglycine t-butyl ester (DAPT) (Tocris®), with medium changed every second day.

### Example 6

### Live Imaging of Primary Neurons in Ultrashort Peptide Hydrogels

Neuronal 3D model was established as described earlier in glass bottom confocal cell culture dishes. Timelapse experiment for 3 days was done using ZEISS™ LSM 880 with Airyscan with 5 minutes intervals setting. During the experiment, the onstage incubator of the confocal microscope was used on 37° C. and CO₂ at 5%.

### Example 7

### Recording the Electrical Avidity of Primary Neurons in the 3D Neuronal Model Using Multielectrode Arrays (MEA)

To confirm the functionality of neurons in the established 3D model, extracellular electrical activity was recorded for 40 days using the MEA2100 multichannel system. Neurons were seeded in 6 well MEA200/30iR-Ti or 60MEA200/30iR-Ti. Electrical activity was recorded after 5 days in vitro (DIV), for 5 minutes every two days. The cells placed under the MEA was supplied with 2V (I˜0.7A) by a linear power supplier (TTi, model QL564) to maintain the temperature at 37° C. during the recordings. The recordings were continued until 40 DIV. In all cases, the signals were acquired with the following parameters: amplifier bandwidth=1-25000 Hz, sampling rate=25 k Samples/s, high pass filter frequency=5 Hz.

### Example 8

### Cell Viability Testing

The viability of 3D cultured cells is assessed using the LIVE/DEAD Viability/Cytotoxicity Kit (ThermoFisher®, USA). In which, calcein acetoxymethyl ester (Calcein-AM)is used to detect viable cells and ethidium homodimer-I (EthD-I) is used to detect dead cells. Cell-laden 3D constructs are washed twice with dulbecco's phosphate-buffered saline (D-PBS). Then a staining solution of 2 μM of Calcein-AM and 4 μM of EthD-1 are added to the 3D cell-laden constructs and incubated for 30 minutes at room temperature. After the incubation period, the staining solution is discarded, and 1× DPBS is added to each well before imaging. Stained cells are imaged with an inverted confocal microscope (Zeiss™ LSM 710 Inverted Confocal Microscope, Germany) or ZEISS™ fluorescent microscope.

The viability of primary mouse cortical neurons is assessed after 24 hours, 2, and 3 days of culture.

### REFERENCES

The following references are referred to above and are incorporated herein by reference:
1. Pacitti, D., Privolizzi, R., & Bax, B. E. (2019). Organs to cells and cells to organoids: the evolution of in vitro central nervous system modelling. Frontiers in Cellular Neuroscience, 13, 129.
2. Murphy, A. R., Laslett, A., O'Brien, C. M., & Cameron, N. R. (2017). Scaffolds for 3D in vitro culture of neural lineage cells. Acta Biomaterialia, 54, 1-20.
3. Susapto, H. H., Alhattab, D., Abdelrahman, S., Khan, Z., Alshehri, S., Kahin, K., . . . & Hauser, C. A. (2021). Ultrashort Peptide Bioinks Support Automated Printing of Large-Scale Constructs Assuring Long-Term Survival of Printed Tissue Constructs. Nano Letters, 21(7), 2719-2729.
4. Blakely, B. D., Bye, C. R, Fernando, C. V., Home, M. K, Macheda, M. L., Stacker, S. A., . . . & Parish, C. L. (2011). Wnt5a regulates midbrain dopaminergic axon growth and guidance. PIoS One, 6(3), e18373

All documents, patents, journal articles and other materials cited in the present application are incorporated herein by reference.

While the present disclosure has been disclosed with references to certain embodiments, numerous modifications, alterations, and changes to the described embodiments are possible without departing from the sphere and scope of the present disclosure, as defined in the appended claims. Accordingly, it is intended that the present disclosure not be limited to the described embodiments, but that it has the full scope defined by the language of the following claims, and equivalents thereof.

What is claimed is:

1. A 3-dimensional neuronal tissue model comprising:
live neuronal cells; and
ultrashort self-assembling peptide scaffolds.

2. The 3-dimensional neuronal tissue model of claim 1, wherein the ultrashort self-assembling peptideaving a general formula selected from:
AnBmX, BmAnX, XAnBm and XBmAn
wherein the total number of amino acids of the ultrashort peptide does not exceed 7 amino acids;
wherein A is an aliphatic amino acids, selected from the group consisting of: isoleucine, leucine or any combination thereof, with n being an integer being selected from 0-5;
wherein B is comprised of at least one aromatic amino acid selected from the group consisting of: tyrosine, tryptophan, phenylalanine, hydrophobic amino acid phenylalanine, or comprised of a peptidomimetic amino acid that is the aliphatic counterpart of the aromatic amino acid, such as cyclohexylalanine, which is the counterpart of amino acid phenylalanine with m being an integer being selected from 0-3.
wherein X is comprised of a polar amino acid, selected from the group consisting of: aspartic acid, glutamic acid, lysine, arginine, histidine, cysteine, serine, threonine, asparagine, and glutamine.

3. The 3-dimensional neuronal tissue model of claim 1, wherein the ultrashort self-assembling peptide made of the scaffolds is selected from the group consisting of IIFK, IIZK, FFIK, IVFR, IVZR and KIVAV.

4. The 3-dimensional neuronal tissue model of claim 1, wherein the concentration of ultrashort self-assembling peptide used to form the scaffolds is at least 1 mg/ml.

5. The 3-dimensional neuronal tissue model of claim 1, wherein the rigidity of the peptide scaffold is at least 5 kPa.

6. The 3-dimensional neuronal tissue model of claim 1, wherein the density of the neuronal cells within the model is at least 1,000 cells/μl peptide hydrogel construct.

7. The 3-dimensional neuronal tissue model of claim 7, wherein the peptide scaffold supports the proliferation of neurons, as well as growth of neurites and branches.

8. The 3-dimensional neuronal tissue model of claim 1, wherein viability of neurons is at least 90%.

9. The 3-dimensional neuronal tissue model of claim 1, wherein extracellular electrical activities are detectable using electrode-based detection methods, wherein the electrode-based detection method is the multielectrode arrays (MEA)

10. An in vitro drug testing platform comprising of the 3-dimensional neuronal tissue model of claim 1.

11. A tool for studying neurological disorders comprising of the 3-dimensional neuronal tissue model of claim 1.

12. A surgical implant comprising the 3-dimensional neuronal tissue model of claim 1, wherein the surgical implant is used in cellular replacement therapies for neurodegenerative diseases, brain cancer, traumatic brain injuries or other neurological disorders.

13. A 3-dimensional neuronal tissue model comprising:
live embryonic stem cells (ESCs); and
ultrashort self-assembling peptide scaffolds,
wherein the ESCs are capable of differentiating into neurons.

14. The 3-dimensional neuronal tissue model of claim 13, wherein the neurons express at least one of neuron-specific β-III tubulin (TUJ1) and T-Box Brain Transcription Factor 1 (TBR1).

15. The 3-dimensional neuronal tissue model of claim 13, wherein the density of the ESCs within the model is at least 60 cells/μl peptide hydrogel construct.

16. The 3-dimensional neuronal tissue model of claim 13, wherein the ESCs are capable of differentiating into neurons in 33 days or less.

17. A method of creating a 3-dimensional neuronal tissue model comprising:
suspending neurons in tissue culture media;
dissolving an ultrashort self-assembling peptide in buffer solution;
loading a 3D bioprinter with the suspended neurons and peptide solution;
printing the 3-dimensional neuronal tissue model using the 3D bioprinter; and
3D culturing the neurons by keeping the 3-dimensional neuronal tissue model in culture media.

18. A method of creating a 3-dimensional neuronal tissue model comprising:
suspending ESCs in tissue culture media;
dissolving an ultrashort self-assembling peptide in buffer solution;
loading a 3D bioprinter with the suspended ESCs and peptide solution;
creating the 3-dimensional neuronal tissue model using the 3D bioprinter or manually; and
3D culturing the ESCs by keeping the 3-dimensional neuronal tissue model in culture media,
wherein growth media supplements are added during the creation of the 3-dimensional neuronal tissue model.

19. The method of claim 18, wherein the growth media supplements is at least one selected from the group consisting of leukemia inhibitory factor (LIF), LDN193189, GlutaMAX™, non-essential amino acids (NEAA), insulin-transferrin-selenium-sodium pyruvate supplement (ITS-A), beta-mercaptoethanol, N2 supplement, vitamin B27, vitamin A, Sonic Hedgehog (Shh), fibroblast growth factor 8 (FGF8), glycogen synthase kinase 3 inhibitor, fibroblast growth factor 2 (FGF2), glial cell-derived neurotrophic factor (GDNF), brain-derived neurotrophic factor (BDNF), γ-secretase inhibitor and purmorphamine (PM).
